(19) United States
(12) Patent Application Publication
(10) Pub. No.: US 2026/0317752 A1
(43) Pub. Date: Oct. 15, 2026

(54) METHOD FOR OPERATING MICROBIAL CO-IMMOBILIZED FILLER PROCESSING APPARATUS

(71) Applicant: Ocean University of China, Qingdao (CN)

(72) Inventors: Moxi XUE, Qingdao (CN); Fengmin LI, Qingdao (CN); Qian WANG, Qingdao (CN); Yuanyuan LI, Qingdao (CN); Jielin LI, Qingdao (CN)

(21) Appl. No.: 19/380,878

(22) Filed: Nov. 5, 2025

(30) Foreign Application Priority Data
Feb. 28, 2025 (CN) 202510236906.1

(51) Int. Cl. C12N 11/14

(52) U.S. Cl. CPC C12N 11/14

(57) ABSTRACT

A method for operating a microbial co-immobilization filler processing apparatus, including sequential cleaning, drying, embedding, recovery, and stabilization steps under comprehensive monitoring and precise control. Controlled washing and drying parameters remove impurities and moisture, providing a surface suitable for microbial attachment. During recovery, a high-viscosity pump recirculates excess embedding agent to a reagent storage tank to minimize material loss. The apparatus performs real-time quality monitoring of the embedding agent, and upon detection of a nonconforming condition, automatically initiates a cleaning and replenishment cycle to maintain consistent quality. The resulting microbial co-immobilization filler demonstrates stable and efficient nitrogen removal performance in wastewater treatment applications.

### CROSS-REFERENCE TO RELATED APPLICATIONS

Pursuant to 35 U.S.C. § 119 and the Paris Convention Treaty, this application claims foreign priority to Chinese Patent Application No. 202510236906.1 filed Feb. 28, 2025, the contents of which, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, MA 02142.

### BACKGROUND

The disclosure relates to the field of wastewater treatment technology, and more particularly, to a method for operating a microbial co-immobilized filler processing apparatus.

Microbial immobilization technology has advanced rapidly, and microbial co-immobilized fillers have found increasing applications in environmental remediation, bioreactors, and various industrial processes. However, existing technologies and apparatuses for producing microbial co-immobilized filler exhibit several limitations. Conventional apparatuses often fail to provide real-time monitoring of the quality of embedding reagents, which directly affects the stability and performance of the resulting fillers in subsequent applications. In addition, the inability to promptly remove degraded reagents or to control the cleanliness of key apparatus components may result in contamination with pollutants or culture liquids, thereby compromising the microbial immobilization process and degrading filler performance. Furthermore, conventional apparatuses also lack mechanisms to maintain optimal cleanliness and reagent quality throughout the production process, resulting in reduced production efficiency and inconsistent product quality. Accordingly, there remains a need for an improved method of operation that enables real-time monitoring of apparatus cleanliness, ensures the quality of embedding reagents, and maintains a hygienic production environment, thereby enhancing both the production efficiency and the product quality of microbial co-immobilized fillers.

### SUMMARY

To solve the aforesaid problems, an objective of the disclosure is to provide a method for operating a microbial co-immobilized filler processing apparatus. The apparatus comprises a first conveyor belt, a feed hopper, a cleaning assembly, a first drying assembly, an embedding assembly, at least one stabilization tank, a second conveyor belt, a traction mechanism, a second drying assembly, a discharge hopper, and an intelligent control unit. The feed hopper is disposed upstream of the first conveyor belt. The cleaning assembly is disposed above the first conveyor belt. The first drying assembly is disposed downstream of the cleaning assembly. The embedding assembly is disposed downstream of the first drying assembly. The at least one stabilization tank is disposed downstream of the first conveyor belt. The traction assembly is disposed above the at least one stabilization tank. The second drying assembly is disposed downstream of the at least one stabilization tank. The discharge hopper is disposed downstream of the second drying assembly.

The intelligent control unit comprises a controller, a processor, a first sensor group, a second sensor group, and a third sensor group. The first sensor group comprises a first liquid-level sensor and a first weight sensor. The second sensor group comprises a second liquid-level sensor, a pH sensor, a density sensor, a temperature sensor, and an optical-density sensor. The third sensor group comprises a third liquid-level sensor and a second weight sensor. The controller is configured to automatically adjust operational parameters of the apparatus.

The cleaning assembly comprises a spray mechanism and a water collection tank. The spray mechanism is disposed above the first conveyor belt. The water collection tank is disposed below the first conveyor belt and aligned with the spray mechanism. The embedding assembly comprises a reagent storage tank, a reagent recovery tank, a reagent recovery pipe, and a high-viscosity liquid pump. The reagent storage tank is disposed downstream of the first drying assembly. The reagent recovery tank is disposed below a downstream end of the first conveyor belt. The reagent recovery pipe and the high-viscosity liquid pump are fluidly connected between the reagent storage tank and the reagent recovery tank. A scraper is disposed above the reagent recovery tank and in contact with a surface of the first conveyor belt.

The first liquid-level sensor and the first weight sensor are disposed within the water collection tank and configured to monitor a first liquid level and first liquid weight therein for calculating a first liquid density. The second liquid-level sensor, the pH sensor, the density sensor, the temperature sensor, and the optical-density sensor are disposed within the reagent storage tank and configured to monitor a second liquid level, pH, a second liquid density, temperature, and optical density of an embedding reagent therein. The third liquid-level sensor and the second weight sensor are disposed within the reagent recovery tank and configured to monitor a third liquid level and a second liquid weight therein for calculating a third liquid density. The intelligent control unit further comprises a fourth liquid-level sensor disposed within the at least one stabilization tank and configured to monitor a fourth liquid level therein;
the method comprises:
S1. loading filler carriers into the feed hopper and starting the apparatus to convey the filler carriers along the first conveyor belt; cleaning the filler carriers by the spray mechanism and allowing wastewater generated therefrom to flow into the water collection tank; calculating, by the processor, the first liquid density in the water collection tank based on data received from the first sensor group; and adjusting, by the controller, a spray condition when the first liquid density exceeds a first density threshold, or discharging wastewater when the first liquid level exceeds a first level threshold;
S2. transferring, by the first conveyor belt, the cleaned filler carriers to the first drying assembly for drying and conveying the dried filler carriers beneath the reagent storage tank; spraying an embedding reagent from the reagent storage tank onto the dried filler carrier; determining, by the processor, whether a quality index Z of the embedding reagent falls outside a preset threshold range, or whether the second liquid level in the reagent storage tank falls below a second level threshold; and executing, by the controller, a cleaning operation on the reagent storage tank when the quality index Z of the embedding reagent falls outside the preset threshold range, or replenishing the reagent storage tank when the second liquid level falls below the second level threshold;
S3. conveying, by the first conveyor belt, the sprayed filler carriers into the at least one stabilization tank; determining, by the processor, whether the fourth liquid level of the at least one stabilization tank connected to the downstream end of the first conveyor belt exceeds a third level threshold; and replacing the at least one stabilization tank when the liquid level exceeds the third level threshold;
S4. scraping, by the scraper, excess embedding reagent remaining on the first conveyor belt into the reagent recovery tank after the sprayed filler carriers enter the stabilization tank; pumping, by the high-viscosity liquid pump, the embedding reagent recovered from the reagent recovery tank to the reagent storage tank; calculating, by the processor, the third liquid density in the reagent recovery tank; and initiating, by the controller, a cleaning operation of the reagent recovery tank when the third liquid density exceeds a second density threshold;
S5. discharging liquid from the at least one stabilization tank after stabilization of the sprayed filler carriers to obtain stabilized filler, transferring the stabilized filler via the traction assembly onto the second conveyor belt, and drying the stabilized filler in the second drying assembly; and
S6. obtaining the dried stabilized filler as a finished microbial co-immobilized filler product and conveying the finished product to the discharge hopper.

In a class of this embodiment, in S2, the quality index Z of the embedding reagent is calculated according to the formula:

Z=5·C₁+1·C₂+8·C₃+0.3·C₄
wherein C₁ is the optical density of the embedding reagent, C₂ is the pH of the embedding reagent, C₃ is the second liquid density, and C₄ is the temperature of the embedding reagent; and a threshold range for the quality index Z is set in a range from 32 to 38. The controller is configured to initiate the cleaning operation of the reagent storage tank when the calculated quality index Z falls outside the threshold range.

In a class of this embodiment, a threshold value for the quality index Z is defined as 35, and the cleaning operation on the reagent storage tank is initiated when the quality index Z falls outside the threshold value.

In a class of this embodiment, in S1, the first density threshold is set within a range from 1.8 g/cm³ to 1.9 g/cm³.

In a class of this embodiment, in S1, the first density threshold is set to 1.9 g/cm³.

In a class of this embodiment, in S1, adjusting the spray conditions comprises draining the water collection tank, increasing a spray water flow rate, reducing a conveying speed of the first conveyor belt, or a combination thereof.

In a class of this embodiment, the spray water flow rate is set within a range from 1.5 L/s to 1.8 L/s, and the conveying speed of the first conveyor belt is set within a range from 4 m/min to 8 m/min.

In a class of this embodiment, in S4, the third density threshold is set in a range from 1.05 g/cm³ to 1.1 g/cm³.

In a class of this embodiment, in S4, the third density threshold is set to 1.05 g/cm³.

In a class of this embodiment, the first level threshold in S1 is set to 0.4 m; the second level threshold in S2 is set to 0.15 m; and the third level threshold in S3 is set to 1.4 m.

The following advantages are associated with the disclosed method.

The disclosed method provides for the large-scale, engineered production of the microbial co-immobilized filler through comprehensive monitoring and precise control of key processes, including cleaning, drying, embedding, reagent recovery, and stabilization. The method ensures high efficiency and consistency in production. Control of the cleaning water volume and drying temperature removes surface impurities and moisture from the filler carriers, thereby establishing optimal conditions for microbial attachment and growth. The high-viscosity liquid pump turns the recovered embedding reagent to the reagent storage tank, which minimizes waste and enhances resource utilization. The method comprises real-time monitoring of the quality of the recovered reagent. Upon detection that the quality deviates from predetermined standards, the reagent storage tank automatically activates a cleaning and replenishment mode. The method maintains the embedding reagent with quality specifications, thereby ensuring the microbial co-immobilized filler deliver stable and highly effective total nitrogen (TN) removal in subsequent wastewater treatment processes. Collectively, the method features a significant enhancement in operational efficiency. When operating for 8 hours per day, the apparatus is capable of processing approximately 7.2 m³ of the filler carriers, requiring only a single operator for apparatus start-up, reagent preparation, and replenishment. By comparison, manual laboratory embedding performed by a single operator over the same period yields me about 0.04 m³ of the filler, indicating an approximate 180-fold improvement in processing efficiency.

### BRIEF DESCRIPTION OF THE DRAWINGS

FIG. 1 is a schematic diagram of a microbial co-immobilized filler processing apparatus according to Example 1 of the disclosure;

FIG. 2 is a flow diagram illustrating operation of a cleaning assembly according to Example 1 of the disclosure;

FIG. 3 is a flow diagram illustrating operation of an embedding assembly according to Example 1 of the disclosure;

FIG. 4 is a flow diagram illustrating operation of a reagent recovery tank according to Example 1 of the disclosure; and

FIG. 5 is a flow diagram illustrating operation of at least one stabilization tank according to Example 1 of the disclosure.

In the drawings, the following reference numbers are used: 1. First conveyor belt; 2. Feed hopper; 3. Cleaning assembly; 4. First drying assembly; 5. Embedding assembly; 6. Stabilization tank; 7. Traction mechanism; 8. Second conveyor belt; 9. Second drying assembly; 10. Discharge hopper; 11. Rotary base; 101. Second baffle; 301. Spray mechanism; 302. Water collection tank; 303. First inlet pipe; 304. First drain pipe; 501. Reagent storage tank; 502. Reagent recovery tank; 503. Reagent recovery pipe; 504. High-viscosity liquid pump; 505. Second drain pipe; 506. Distribution valve; 507. Third drain pipe; 508. Second inlet pipe; 509. First baffle; 510. Scraper; 601. Suspension hole; 602. Fourth drain pipe; 701. Traction wire; 702. Stop block; 901. First liquid-level sensor; 902. First weight sensor; 903. Second liquid-level sensor; 904. PH sensor; 905. Optical-density sensor; 906. Density sensor; 907. Temperature sensor; 908. Third liquid-level sensor; 909. Second weight sensor; and 910. Fourth liquid-level sensor.

### DETAILED DESCRIPTION

To further illustrate the disclosure, embodiments detailing the method for operating the microbial co-immobilized filler processing apparatus are described below. It should be noted that the following embodiments are intended to describe and not to limit the disclosure.

### Example 1

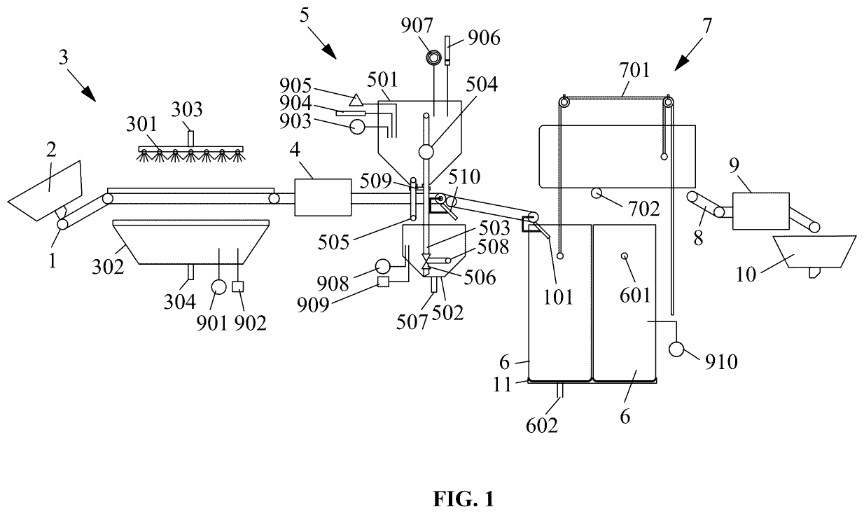

This embodiment provides a method for operating a microbial co-immobilized filler processing apparatus. As illustrated in FIG. 1, the apparatus comprises a first conveyor belt 1, a feed hopper 2, a cleaning assembly 3, a first drying assembly 4, an embedding assembly 5, two stabilization tanks 6, a traction mechanism 7, a second conveyor belt 8, a second drying assembly 9, a discharge hopper 10, a rotary base 11, and an intelligent control unit.

The first conveyor belt 1 includes a plurality of staggered chain links, each measuring 1 cm×5 cm, with adjacent chain links spaced apart by a gap of 0.25 to 0.35 cm. The first conveyor belt 1 has a width of 0.5 m, is operated at a speed of 16 m/min, and includes two opposing side waterproof baffles, each having a height of 5 cm.

The feed hopper 2, having a cuboid shape, is disposed upstream of the first conveyor belt 1 and is configured to receive and convey filler carriers onto the conveyor belt.

The cleaning assembly 3 is disposed above the first conveyor belt 1 and comprises a spray mechanism 301, a water collection tank 302, a first inlet pipe 303, and a first drain pipe 304. The spray mechanism 301 is configured to spray water over the filler carriers and covers a cleaning area of 1 m×0.5 m. The water collection tank 302 is disposed below the first conveyor belt 1 and aligned with the spray mechanism 301 to collect wastewater generated during cleaning, with a cuboid bottom measuring 1 m×0.5 m, a top measuring 1.5 m×0.5 m, and an effective volume of 232 L. The first inlet pipe 303 supplies water to the spray mechanism 301, and the water collection tank 302 is connected to the first drain pipe 304.

The first drying assembly 4 is disposed downstream of the cleaning assembly 3 and includes a tunnel heating furnace with a channel dimension of 1.5 m ×0.5 m×0.3 m, a maximum operating temperature of 200° C., and a stainless-steel herringbone mesh belt with opposed bent edges for uniformly drying the cleaned filler carriers.

The embedding assembly 5 is disposed downstream of the first drying assembly 4 and includes a reagent storage tank 501, a reagent recovery tank 502, a reagent recovery pipe 503, a high-viscosity liquid pump 504, a second drain pipe 505, a distribution valve 506, a third drain pipe 507, and a second inlet pipe 508. The reagent storage tank 501, having an effective volume of 0.6 m³, is configured to store an embedding reagent and is provided with a discharge port and a first baffle 509. The first baffle 509 is configured to open and close the discharge port. The discharge port is disposed 3 cm above the first conveyor belt 1 and has a length of 0.5 m in length and a width of 0.5 cm. The reagent recovery tank 502, having a cuboid bottom and an effective volume of 0.2 m³, is disposed beneath the downstream end of the first conveyor belt 1 to collect excess embedding reagent. The reagent recovery tank 502 is connected to the third drain pipe 507 and a second inlet pipe 508. A scraper 510 is disposed above the recovery tank and in contact with a surface of the first conveyor belt 1 to remove excess embedding reagent. The reagent recovery pipe 503 and the high-viscosity liquid pump 504 are disposed between the reagent storage tank 501 and the reagent recovery tank 502 to form a recirculation path. The high-viscosity liquid pump 504 is disposed on the reagent recovery pipe 503. A first end of the reagent recovery pipe 503 is connected to the second inlet pipe 508 and the reagent recovery tank 502 via the distribution valve 506, while a second end of the reagent recovery pipe 503 is connected to the reagent storage tank 501.

The two stabilization tanks 6 are disposed downstream of the first conveyor belt 1. The two stabilization tanks 6 are mounted on the rotary base 11 and configured to be alternatively aligned with the downstream end of the first conveyor belt. The downstream end of the first conveyor belt 1 includes a second baffle 101. Each stabilization tank 6 has internal dimensions of 0.5 m×0.5 m×1.5 m and is configured to contain a 4% calcium chloride solution. Each stabilization tank 6 includes a serrated docking section for contacting the downstream end of the first conveyor belt 1. Each stabilization tank 6 further includes a sidewall provided with a suspension hole 601 and connected to a fourth drain pipe 602.

The traction mechanism 7 is disposed downstream of the two stabilization tanks 6 and comprises a traction wire 701 and a stop block 702. The second conveyor belt 8 and the second drying assembly 9 are disposed downstream of the traction mechanism 7. The second drying assembly 9 is a tunnel heating furnace having the same channel dimensions and maximum operating temperature as the first drying assembly 4. The second drying assembly 9 includes a stainless-steel herringbone mesh belt with opposed bent edges.

The discharge hopper 10 is disposed downstream of the second drying assembly 9.

The intelligent control unit includes a controller, a processor, a first liquid-level sensor 901, a first weight sensor 902, a second liquid-level sensor 903, a pH sensor 904, an optical-density sensor 905, a density sensor 906, a temperature sensor 907, a third liquid-level sensor 908, a second weight sensor 909, and a fourth liquid-level sensor 910. The first liquid-level sensor 901 and first weight sensor 902 are disposed in the water collection tank 302 and. The second liquid-level sensor 903, the pH sensor 904, the optical-density sensor 905, the density sensor 906, and the temperature sensor 907 are mounted in the reagent storage tank 501. The third liquid-level sensor 908 and second weight sensor 909 are disposed in the reagent recovery tank 502. The fourth liquid-level sensor 910 is positioned within the stabilization tank 6 aligned with the downstream end of the first conveyor belt 1.

The filler carriers are selected from smooth-surfaced materials such as ceramsite, gravel, or steel slag. The performance parameters of the filler carriers comply with the standard CJ/T43-2005, Filter Materials For Water Treatment. The embedding reagent is prepared by mixing an aerobic denitrifying bacterial culture with a gelling agent. The gelling agent is selected from the group consisting of agar, sodium alginate, and carrageenan, preferably at a concentration of 4% (w/w). A solution of the gelling agent is sterilized at 73.5 kPa for 5 min and cooled to room temperature. The aerobic denitrifying bacterial culture is added to the solution of the gelling agent at a concentration of 5-15 mg/L. The aerobic denitrifying bacteria are enriched from commercial bacterial preparations using a batch aeration reactor. The enrichment procedure is performed in the following sequence: influent is introduced, followed by aeration, then stirring, then settling for 0.3 h, and finally effluent removal for 0.15 h. The initial aeration time is 12 hours. Thereafter, the aeration duration is increased by 1-2 hours every three days, with a corresponding reduction in stirring time. After 28 days, acclimation is considered complete upon the appearance of light gray, flocculent sludge, indicating good microbial health. During the initial acclimation period, an FM culture medium is used as the influent, and a proportion of DM culture medium is then gradually increased to acclimate the microorganisms to the new environment. The stepwise adjustment facilitates the selection of the most adaptable strains, thereby enriching a target microbial community. The FM culture medium is provided, comprising, per liter of medium: 1.0 g of KNO₃, 1.0 g of beef extract, and 5.0 g of peptone. The DM culture medium is provided, comprising, per liter of medium: 7.9 g of Na₂HPO₄⋅7H₂O, 1.5 g of KH₂PO₄, 0.3 g of NH₄Cl, 0.1 g of MgSO₄⋅7H₂O, 4.7 g of sodium succinate, 2 mL of trace element solution, and a nitrogen source selected from the group consisting of KNO₃ and NaNO₂. The trace element solution comprises, per liter of solution: 50.0 g of EDTA, 2.2 g of ZnSO₄, 5.5 g of CaCl₂, 5.06 g of MnCl₂⋅4H₂O, 5.0 g of FeSO₄⋅7H₂O, 1.1 g of (NH₄)₆Mo₇O₂⋅4H₂O, 1.57 g of CuSO₄⋅5H₂O, and 1.61 g of CoCl₂⋅6H₂O, and is adjusted to a pH of 7.0.

The method for operating the microbial co-immobilized filler processing apparatus includes the following steps:

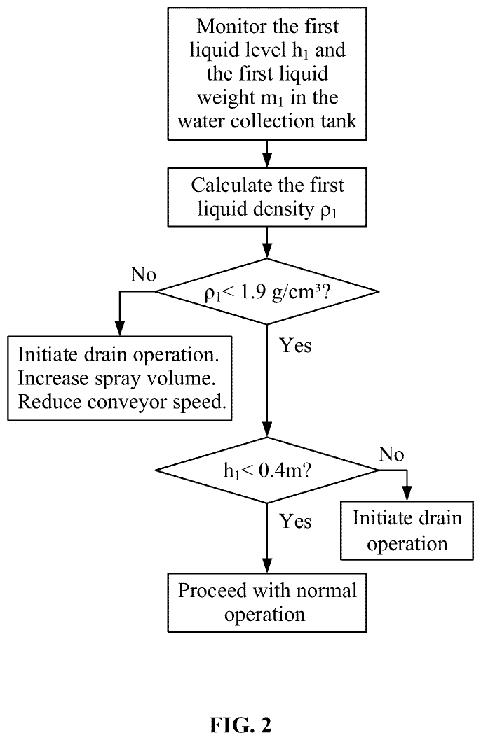

S1: Filler carriers are loaded into the feed hopper 2. The apparatus is then started, and the filler carriers are conveyed from the feed hopper 2 onto the first conveyor belt 1 operating at a speed of 16 m/min. The spray mechanism 301 is configured to clean the filler carriers on the first conveyor belt 1 using a spray water flow rate of 1.0 L/s. The resulting wastewater flows into the water collection tank 302. As shown in FIG. 2, the first liquid-level sensor 901 and the first weight sensor 902 respectively measure the monitor the first liquid height (h₁) and first liquid weight (m₁) in the water collection tank 302. The data are transmitted to the processor. The processor calculates the first liquid density (ρ₁) and analyzes apparatus conditions.

When ρ₁<1.9 g/cm³ and h₁<0.4 m, the controller issues no command.

When ρ₁<1.9 g/cm³ and h₁≥0.4 m, the controller activates the first drain pipe 304 to discharge wastewater.

When ρ₁≥1.9 g/cm³, the controller activates the first drain pipe 304 to discharge wastewater, increases the spray water flow rate from 1.0 L/s to 1.5 L/s, and reduces the speed of the first conveyor belt 1 from 16 m/min to 4-8 m/min.

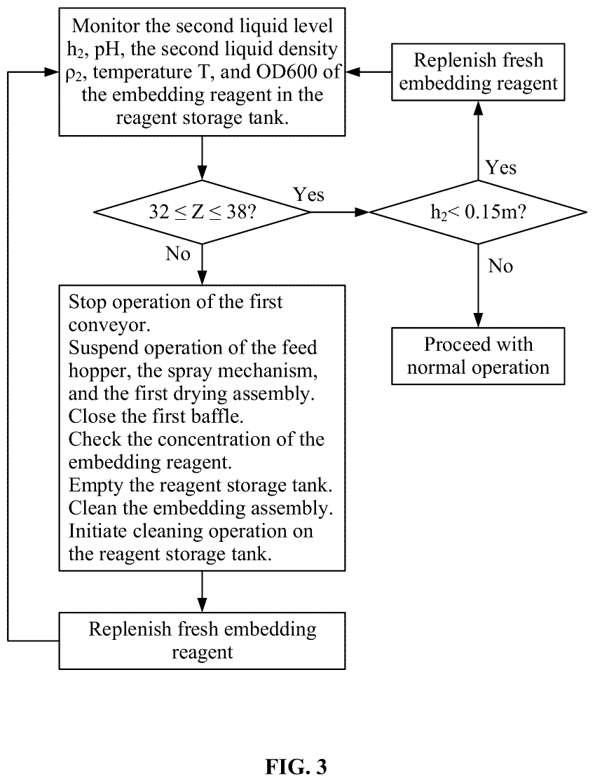

S2: The cleaned filler carriers are transported into the first drying assembly 4. After drying, the filler carriers are conveyed beneath the reagent storage tank 501, and the embedding reagent is sprayed onto the dried filler carriers. As shown in FIG. 3, the second liquid-level sensor 903, the pH sensor 904, the optical-density sensor 905, the density sensor 906, and the temperature sensor 907 measure, respectively, the second liquid height (h₂), the pH, the optical density (OD₆₀₀), the second liquid density (ρ₂), and the temperature (T) of the embedding reagent in the reagent storage tank. The measured data are transmitted to the processor and used to calculate the quality index Z using the following formula:

Z=5·C₁+1·C₂+8·C₃+0.3·C₄
where C₁ represents the optical density (OD₆₀₀) of the embedding reagent, C₂ represents the pH of the embedding reagent, C₃ represents the second liquid density (ρ₂), and C₄ represents the temperature (T) of the embedding reagent. The threshold range for the quality index Z is set to 32-38.

When 32≤Z≤38 and h₂>0.15 m, the controller issues no command.

When 32≤Z≤38 and h₂≤0.15 m, the controller issues a refill alert for the embedding reagent.

When Z>38 or Z<32, the controller: (i) stops operation of the first conveyor 1; (ii) suspends operation of the feed hopper 2, the spray mechanism 301, and the first drying assembly 4; (iii) checks the concentration of the embedding reagent and closes the first baffle 509; (iv) drains the embedding reagent from the reagent storage tank 501 through the second drain pipe 505; (v) adjusts the distribution valve 506 and introduces 70° C. hot water from the second inlet pipe 508 into the reagent recovery pipe 503; (vi) delivers the 70° C. hot water into the reagent storage tank 501 using the high-viscosity liquid pump 504 for cleaning; (vii) drains the resulting wastewater through the second drain pipe 505; and (viii) initiates replenishment of fresh embedding reagent.

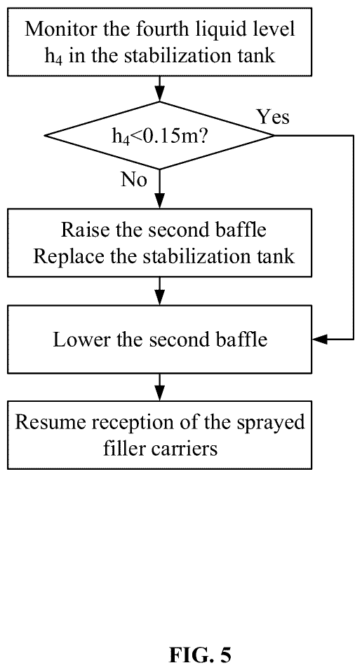

S3: The spayed filler carriers pass through the second baffle 101 and the serrated docking section into one of the two stabilization tanks 6 aligned with the downstream end of the first conveyor belt 1. As shown in FIG. 5, the fourth liquid-level sensor 910 monitors the fourth liquid height (h₄) in each stabilization tank 6 and transmits the data to the processor:

When h₄<1.40 m, the controller maintains the second baffle 101 in a lowered position to receive the spayed filler carriers.

When h₄≥1.40 m, the controller raises the second baffle 101, actuates the rotary base 11 to replace the stabilization tank 6, and then lowers the second baffle 101 to resume receiving the spayed filler carriers.

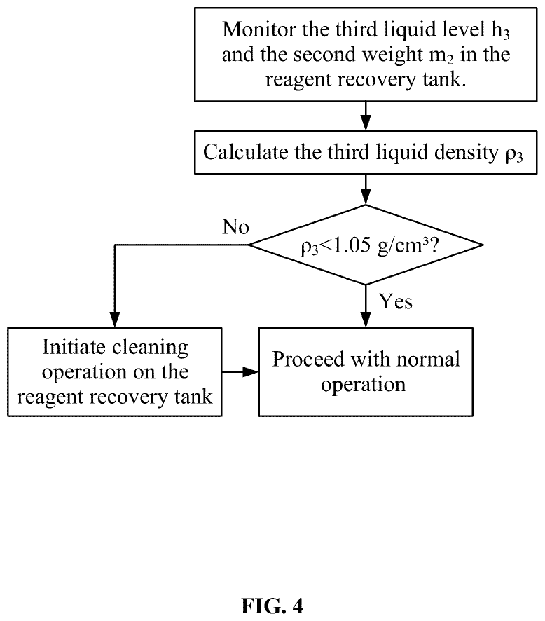

S4: Once the spayed filler carriers enter the stabilization tank 6, the scraper 510 removes excess embedding reagent from the first conveyor belt 1 into the reagent recovery tank 502. The distribution valve 506 is adjusted, and the high-viscosity liquid pump 504 is activated to pump the recovered embedding reagent back into the reagent storage tank 501. As shown in FIG. 4, the third liquid-level sensor 908 and the second weight sensor 909 measure, respectively, the third liquid height (h₃) and the second liquid weight (m₂) in the reagent recovery tank 502. The processor calculates the third liquid density (ρ₃) of the recovered embedding reagent.

When ρ₃<1.05 g/cm³, no action is required.

When ρ₃≥1.05 g/cm³, the controller stops the high-viscosity pump 504, adjusts the distribution valve 506, introduces 70° C. hot water through the second inlet pipe 508 to clean the reagent recovery tank 502, and discharges wastewater through the third drain pipe 507.

S5: After a 2-hour stabilization period, the liquid in the stabilization tank 6 is drained via the fourth drain pipe 602. The traction mechanism 7 engages the suspension hole 601 to lift the stabilization tank 6 to a height of 1.0 m. The traction mechanism 7 then moves the stabilization tank 6 laterally, and contact with a stop block 702 causes the stabilization tank 6 to tilt, discharging the stabilized filler onto the second conveyor belt 8.

S6: The second conveyor belt 8 conveys the stabilized filler to the second drying assembly 9. After drying, the final microbial co-immobilized filler product is discharged through the discharge hopper 10 for subsequent collection or processing.

When operating for 8 hours per day, the apparatus is capable of processing approximately 7.2 m³ of the filler carriers, requiring only a single operator for apparatus start-up, reagent preparation, and replenishment. By comparison, manual laboratory embedding performed by a single operator over the same period yields only about 0.04 m³ of the filler, indicating an approximate 180-fold improvement in processing efficiency.

The microbial co-immobilized filler particles have an average diameter of approximately 3 cm. When utilized to construct a subsurface flow wetland system with a depth of 1.0 m and a diameter of 30 cm for treatment of Class 1B influent, a 24-hour static denitrification test shows total nitrogen (TN) removal efficiencies ranging from 49.86 to 93.16%.

The disclosed method may be implemented to operate an apparatus for preparing the microbial co-immobilized filler used in subsurface flow wetland system. Such filler may be applied to enhance denitrification, promote phosphorus removal, improve sediment conditions, or suppress algal blooms within watershed environments.

### Example 2

Example 2 is similar to Example 1, except that the threshold value of the quality index Z for the embedding reagent is set to 35.

The finished microbial co-immobilized filler product has a particle diameter of about 3 cm and exhibited enhanced nitrogen removal performance. After 24 hours of static denitrification, the total nitrogen (TN) removal rate ranges from 77.18-93.16%, which is higher than the range of 49.86-93.16% observed in Example 1.

It will be obvious to those skilled in the art that changes and modifications may be made, and therefore, the aim in the appended claims is to cover all such changes and modifications.

What is claimed is:

1. A method for operating a microbial co-immobilization filler processing apparatus, the apparatus comprising a first conveyor belt, a feed hopper, a cleaning assembly, a first drying assembly, an embedding assembly, at least one stabilization tank, a second conveyor belt, a traction mechanism, a second drying assembly, a discharge hopper, and an intelligent control unit;
wherein:
the feed hopper is disposed upstream of the first conveyor belt; the cleaning assembly is disposed above the first conveyor belt; the first drying assembly is disposed downstream of the cleaning assembly; the embedding assembly is disposed downstream of the first drying assembly; the at least one stabilization tank is disposed downstream of the first conveyor belt; the traction assembly is disposed above the at least one stabilization tank; the second drying assembly is disposed downstream of the at least one stabilization tank; and the discharge hopper is disposed downstream of the second drying assembly;
the intelligent control unit comprises a controller, a processor, a first sensor group, a second sensor group, and a third sensor group; the first sensor group comprises a first liquid-level sensor and a first weight sensor; group comprises a second liquid-level sensor, a pH sensor, a density sensor, a temperature sensor, and an optical-density sensor; the third sensor group comprises a third liquid-level sensor and a second weight sensor; the intelligent control unit further comprises a fourth liquid-level sensor; and the controller is configured to automatically adjust operational parameters of the apparatus;
the cleaning assembly comprises a spray mechanism and a water collection tank; the spray mechanism is disposed above the first conveyor belt; and the water collection tank is disposed below the first conveyor belt and aligned with the spray mechanism;
the embedding assembly comprises a reagent storage tank, a reagent recovery tank, a reagent recovery pipe, and a high-viscosity liquid pump; the reagent storage tank is disposed downstream of the first drying assembly; the reagent recovery tank is disposed below a downstream end of the first conveyor belt; the reagent recovery pipe and the high-viscosity liquid pump are fluidly connected between the reagent storage tank and the reagent recovery tank; and a scraper is disposed above the reagent recovery tank and in contact with a surface of the first conveyor belt;
the first liquid-level sensor and the first weight sensor are disposed within the water collection tank and configured to respectively monitor a first liquid level and a first liquid weight therein for calculating a first liquid density; the second liquid-level sensor, the pH sensor, the density sensor, the temperature sensor, and the optical-density sensor are disposed within the reagent storage tank and configured to monitor a second liquid level, a pH, a second liquid density, a temperature, and an optical density of an embedding reagent therein; the third liquid-level sensor and the second weight sensor are disposed within the reagent recovery tank and configured to respectively monitor a third liquid level and a second liquid weight therein for calculating a third liquid density; and the fourth liquid-level sensor is disposed within the at least one stabilization tank and configured to monitor a fourth liquid level therein;
the method comprises:
S1. loading filler carriers into the feed hopper and starting the apparatus to convey the filler carriers along the first conveyor belt; cleaning the filler carriers by the spray mechanism and allowing wastewater generated therefrom to flow into the water collection tank; calculating, by the processor, the first liquid density in the water collection tank based on data received from the first sensor group; and adjusting, by the controller, a spray condition when the first liquid density exceeds a first density threshold, or discharging the wastewater when the first liquid level exceeds a first level threshold;
S2. transferring, by the first conveyor belt, the cleaned filler carriers to the first drying assembly for drying and conveying the dried filler carriers beneath the reagent storage tank; spraying an embedding reagent from the reagent storage tank onto the dried filler carrier; determining, by the processor, whether a quality index Z of the embedding reagent falls outside a preset threshold range, or whether the second liquid level in the reagent storage tank falls below a second level threshold; and executing, by the controller, a cleaning operation on the reagent storage tank when the quality index Z of the embedding reagent falls outside the preset threshold range, or replenishing the reagent storage tank when the second liquid level falls below the second level threshold;
S3. conveying, by the first conveyor belt, the sprayed filler carriers into the at least one stabilization tank; determining, by the processor, whether the fourth liquid level of the at least one stabilization tank exceeds a third level threshold; and replacing the at least one stabilization tank when the liquid level exceeds the third level threshold;
S4. scraping, by the scraper, excess embedding reagent remaining on the first conveyor belt into the reagent recovery tank after the sprayed filler carriers enter the stabilization tank; pumping, by the high-viscosity liquid pump, the embedding reagent recovered from the reagent recovery tank to the reagent storage tank; calculating, by the processor, the third liquid density in the reagent recovery tank; and initiating, by the controller, a cleaning operation of the reagent recovery tank when the third liquid density exceeds a second density threshold;
S5. discharging liquid from the at least one stabilization tank after stabilization of the sprayed filler carriers to obtain stabilized filler, transferring the stabilized filler via the traction assembly onto the second conveyor belt, and drying the stabilized filler in the second drying assembly; and
S6. obtaining the dried stabilized filler as a finished microbial co-immobilized filler product and conveying the finished product to the discharge hopper.

2. The method of claim 1, wherein in S2, the quality index Z of the embedding reagent is calculated according to the formula:
Z=5·C₁+1·C₂+8·C₃+0.3·C₄
C₁ is the optical density of the embedding reagent, C₂ is the pH of the embedding reagent, C₃ is the second liquid density, and C₄ is the temperature of the embedding reagent; and a threshold range for the quality index Z is from 32 to 38; the controller is configured to initiate the cleaning operation of the reagent storage tank when the calculated quality index Z falls outside the threshold range.

3. The method of claim 2, wherein a threshold value for the quality index Z is defined as 35.

4. The method of claim 2, wherein in S1, the first density threshold is set within a range from 1.8 g/cm³ to 1.9 g/cm³.

5. The method of claim 4, wherein in S1, the first density threshold is set to 1.9 g/cm³.

6. The method of claim 2, wherein in S1, adjusting the spray condition comprises draining the water collection tank, increasing a spray water flow rate, reducing a conveying speed of the first conveyor belt, or a combination thereof.

7. The method of claim 6, wherein the spray water flow rate is set within a range from 1.5 L/s to 1.8 L/s, and the conveying speed of the first conveyor belt is set within a range from 4 m/min to 8 m/min.

8. The method of claim 2, wherein in S4, the third density threshold is set within a range from 1.05 g/cm³ to 1.1 g/cm³.

9. The method of claim 8, wherein in S4, the third density threshold is set to 1.05 g/cm³.

10. The method of claim 2, wherein:
the first level threshold in S1 is set to 0.4 m;
the second level threshold in S2 is set to 0.15 m; and
the third level threshold in S3 is set to 1.4 m.
